(19) United States
(12) Patent Application Publication
(10) Pub. No.: US 2023/0245331 A1
(43) Pub. Date: Aug. 3, 2023

(54) MEDICAL IMAGE PROCESSING APPARATUS, MEDICAL IMAGE PROCESSING METHOD, AND RECORDING MEDIUM

(71) Applicant: CANON MEDICAL SYSTEMS CORPORATION, Tochigi (JP)

(72) Inventors: Panjie GOU, Beijing (CN); Xiao XUE, Beijing (CN); Shun ZHAO, Beijing (CN); Qilin XIAO, Beijing (CN)

(73) Assignee: CANON MEDICAL SYSTEMS CORPORATION, Tochigi (JP)

(21) Appl. No.: 18/160,364

(22) Filed: Jan. 27, 2023

(30) Foreign Application Priority Data
Jan. 29, 2022 (CN) 202210109999.8

(51) Int. Cl. G06T 7/33; G06T 7/13; A61B 8/00; A61B 8/08

(52) U.S. Cl. CPC G06T 7/337; G06T 7/13; A61B 8/469; A61B 8/5223; A61B 8/463; A61B 8/5238; G06T 2207/10132; G06T 2207/20221; G06T 2207/30081; G06T 2207/10088; G06T 2207/10081

(57) ABSTRACT

A medical image processing apparatus according to the present embodiment includes processing circuitry. The processing circuitry extracts a registration target area corresponding to a registration target organ from an ultrasonic image by acquiring edges of the registration target organ from the ultrasonic image among a plurality of types of medical images including the registration target organ. The processing circuitry calculates the degree of deformation of a plurality of positions in the registration target area. The processing circuitry performs a rigid registration on the types of medical images on the basis of the degree of deformation for each position calculated at the positions and the similarity between the types of medical images at the positions.

### CROSS-REFERENCE TO RELATED APPLICATIONS

This application is based upon and claims the benefit of priority from Chinese Patent Application No. 202210109999.8, filed on Jan. 29, 2022, the entire contents of all of which are incorporated herein by reference.

### FIELD

Embodiments described herein relate generally to a medical image processing apparatus, a medical image processing method, and a recording medium.

### BACKGROUND

General medical images include ultrasonic (US) images, computed tomography (CT) images, magnetic resonance (MR) images, and the like. A medical image processing apparatus needs to perform a registration on different types of medical images according to the needs of different medical practices.

For example, some surgeries and examinations need to be guided by ultrasonic images, but the ultrasonic images have problems such as low contrast and high noise. Therefore, it may be required to fuse ultrasonic images with medical images (for example, MR images) other than the ultrasonic images to more accurately position a certain organ (or site) of the body. The image fusion usually establishes a correspondence relation between a coordinate system of the ultrasonic image and a coordinate system of the MR image by a rigid registration of the ultrasonic image and the MR image.

However, when collecting the ultrasonic image, a probe needs to be held in close contact with a site to be scanned, and in this case, an organ to be detected is likely to be deformed and an image including a deformation may adversely affect the accuracy of rigid registration. Particularly, when the ultrasonic image includes a relatively severe deformation and a part of the contours (edges) of a deformed organ is similar to a part of the contours (edges) of another organ in the MR image, the organ in the ultrasonic image and the another organ in the MR image may be erroneously registered. This may result in rigid registration failures and image fusion failures.

### BRIEF DESCRIPTION OF THE DRAWINGS

FIG. 1 is a block diagram illustrating an example of the configuration of a medical image processing apparatus according to a first embodiment;

FIG. 2 is a flowchart illustrating a process (medical image processing method) performed by the medical image processing apparatus according to the first embodiment;

FIG. 3 is a schematic diagram illustrating the extraction of a prostate area (registration target area) from an ultrasonic image;

FIG. 4 is a schematic diagram illustrating the shape of the prostate area in an MR image;

FIG. 5 is a diagram for explaining an edge unevenness of a prostate in the ultrasonic image;

FIG. 6 is a schematic diagram for explaining the relationship between an edge curvature and a deformation of the prostate;

FIG. 7 is a schematic diagram for explaining the relationship between a probe distance and a deformation in the ultrasonic image;

FIG. 8 is a schematic diagram illustrating a successful example of a registration of the ultrasonic image and the MR image according to the first embodiment;

FIG. 9 is a schematic diagram illustrating a failure example of a registration of the ultrasonic image and the MR image according to a comparative example;

FIG. 10 is a diagram illustrating a display example of registration reliability;

FIG. 11 is a block diagram illustrating an example of the configuration of a medical image processing apparatus according to a second embodiment;

FIG. 12 is a flowchart illustrating a process (medical image processing method) performed by the medical image processing apparatus according to the second embodiment; and

FIG. 13 is a schematic diagram illustrating the relationship between vectors in a deformation vector field and the degree of deformation in the second embodiment.

### DETAILED DESCRIPTION

A medical image processing apparatus according to the present embodiment includes processing circuitry. The processing circuitry extracts a registration target area corresponding to a registration target organ from an ultrasonic image by acquiring edges of the registration target organ from the ultrasonic image among a plurality of types of medical images including the registration target organ. The processing circuitry calculates the degree of deformation of a plurality of positions in the registration target area. The processing circuitry performs a rigid registration on the types of medical images on the basis of the degree of deformation for each position calculated at the positions and the similarity between the types of medical images at the positions.

### First Embodiment

A medical image processing apparatus 1 according to a first embodiment is described below.

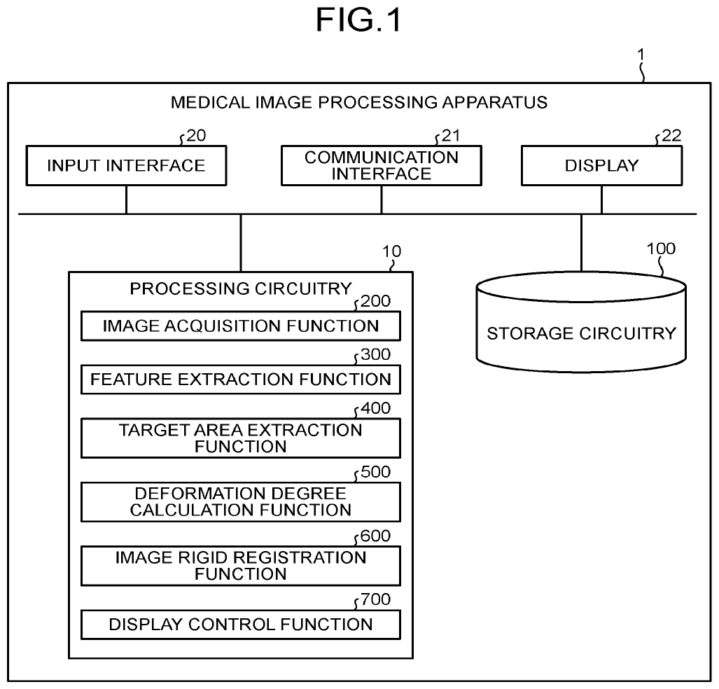

FIG. 1 is a block diagram illustrating an example of the configuration of the medical image processing apparatus 1 according to the first embodiment. As illustrated in FIG. 1, the medical image processing apparatus 1 includes an input interface 20, a communication interface 21, a display 22, processing circuitry 10, and storage circuitry 100.

The input interface 20 is implemented by a trackball, a switch button, a mouse, a keyboard, a touch pad for performing an input operation by touching an operation surface, a touch screen with integrated display screen and touch pad, non-contact input circuitry using an optical sensor, voice input circuitry, or the like, for making various settings. The input interface 20 is connected to the processing circuitry 10, converts an input operation received from an operator into an electrical signal, and outputs the electrical signal to the processing circuitry 10. In FIG. 1, the input interface 20 is provided inside the medical image processing apparatus 1, but may be provided outside the medical image processing apparatus 1.

The display 22 is connected to the processing circuitry 10 and displays various information and various image data output from the processing circuitry 10. For example, the display 22 is implemented by a liquid crystal monitor, a cathode ray tube (CRT) monitor, a touch panel, or the like. For example, the display 22 displays a graphical user interface (GUI) for receiving operator's instructions, various display images, and various processing results by the processing circuitry 10. The display 22 is an example of a display unit. In FIG. 1, the display 22 is provided inside the medical image processing apparatus 1, but may be provided outside the medical image processing apparatus 1. The display 22 is an example of a “display unit”.

The communication interface 21 is a network interface card (NIC) or the like, and communicates with other devices. For example, the communication interface 21 is connected to the processing circuitry 10, and collects image data from an ultrasonic diagnostic apparatus, which is an ultrasonic system, an X-ray computed tomography (CT) apparatus and a magnetic resonance imaging (MRI) apparatus, which are modalities other than the ultrasonic system, and outputs the collected image data to the processing circuitry 10.

The storage circuitry 100 is connected to the processing circuitry 10 and stores therein various data. Specifically, the storage circuitry 100 stores therein at least various medical images for image registration, a fusion image obtained after registration, or the like. For example, the storage circuitry 100 is implemented by a semiconductor memory element such as a random-access memory (RAM) and a flash memory, a hard disk, an optical disc, or the like. The storage circuitry 100 stores therein computer programs corresponding to respective processing functions performed by the processing circuitry 10. In FIG. 1, the storage circuitry 100 is provided inside the medical imaging apparatus 1, but may be provided outside the medical image processing apparatus 1.

The processing circuitry 10 controls each component of the medical image processing apparatus 1 according to an input operation received from the operator via the input interface 20.

For example, the processing circuitry 10 is implemented by a processor. As illustrated in FIG. 1, the processing circuitry 10 has an image acquisition function 200, a feature extraction function 300, a target area extraction function 400, a deformation degree calculation function 500, an image rigid registration function 600, and a display control function 700. Respective processing functions performed by the image acquisition function 200, the feature extraction function 300, the target area extraction function 400, the deformation degree calculation function 500, the image rigid registration function 600, and the display control function 700, which are components of the processing circuitry 10 illustrated in FIG. 1, are stored in the storage circuitry 100 of the medical image processing apparatus 1 in the form of computer programs executable by a computer, for example. The processing circuitry 10 is a processor that reads the computer programs from the storage circuitry 100 and executes the read computer programs, thereby implementing the processing functions corresponding to the executed computer programs. In other words, the processing circuitry 10 in the state of having read the computer programs has the processing functions illustrated in the processing circuitry 10 in FIG. 1.

The term “processor” used in the above description means, for example, circuitry such as a central processing unit (CPU), a graphics processing unit (GPU), an application specific integrated circuit (ASIC), or a programmable logic device (for example, a simple programmable logic device (SPLD), a complex programmable logic device (CPLD), and a field programmable gate array (FPGA)). When the processor is, for example, a CPU, the processor reads out and executes the computer programs stored in the storage circuitry 100 to implement the functions. On the other hand, when the processor is, for example, an ASIC, the computer programs are directly incorporated in the circuitry of the processor instead of storing the computer programs in the storage circuitry 100. Each processor of the present embodiment is not limited to being configured as single piece of circuitry for each processor, and one processor may be configured by combining a plurality of pieces of independent circuitry to implement the functions thereof. The plurality of components in FIG. 1 may be integrated into one processor to implement the functions thereof.

The medical image processing apparatus 1 in FIG. 1 illustrates only functional configurations related to the present embodiment. Although not illustrated in the drawing, the medical image processing apparatus 1 may have other image processing mechanisms. The medical image processing apparatus 1 may also be implemented as a part of the function of an ultrasonic imaging apparatus, a CT imaging apparatus, an MR imaging apparatus, or multifunctional imaging apparatus.

The processing contents of the image acquisition function 200, the feature extraction function 300, the target area extraction function 400, the deformation degree calculation function 500, the image rigid registration function 600, and the display control function 700 performed by the processing circuitry 10 are described below.

The image acquisition function 200 acquires two or more types of medical images as registration target images. The registration target image may be an image acquired from the storage circuitry 100 or an image acquired during surgery by an imaging apparatus such as an ultrasonic imaging apparatus. For example, when performing prostate puncture biopsy surgery, MR images (3D images) of a prostate need to be acquired in advance and stores in the storage circuitry 100 as medical images other than ultrasonic images, and then ultrasonic images (3D images) need to be acquired after the start of surgery.

The feature extraction function 300 extracts image features from the registration target image. Feature maps obtained by extracting the image features are, for example, gradation maps, gradation histograms, dispersion maps, gradient maps, and the like.

The target area extraction function 400 extracts a registration target area corresponding to a registration target organ from an ultrasonic image by acquiring edges of the registration target organ from the ultrasonic image among a plurality of types of medical images including the registration target organ. The extraction can be performed using an edge segmentation algorithm, a deep learning area extraction algorithm, or the like in the related art. The target area extraction function 400 is an example of a “target area extraction unit”.

The deformation degree calculation function 500 calculates the degree of deformation for each position in the registration target area. The “position” in the present embodiment may be a sub-area including one pixel point or a plurality of pixel points in the registration target area. That is, the deformation degree calculation function 500 may calculate the degree of deformation for each pixel point or for each sub-area. The degree of deformation includes at least one of a parameter indicating the unevenness of an edge (that is, edge unevenness parameter), a parameter indicating the smoothness of the edge (that is, edge smoothness parameter), and a parameter indicating distance from an organ to a pressing object (that is, probe distance parameter). The method of calculating the degree of deformation is described later. The deformation degree calculation function 500 is an example of a “deformation degree calculation unit”.

The image rigid registration function 600 performs rigid registration on the types of medical images on the basis of the degree of deformation for each position calculated at the positions and the similarity between the types of medical images at the positions. In the rigid registration process, a position of optimal similarity is found by adjusting the amounts of rotation and translation between the images. To reduce the influence of a deformed portion of the registration target organ on the registration, deformation coefficients are added to a similarity calculation, a large weight is given to an area with a small deformation, and a small weight is given to an area with a large deformation. The method of calculating the similarity is described later. The image rigid registration function 600 is an example of an “image rigid registration unit”.

The display control function 700 causes the display 22 to display a fusion image representing a registration result that is a result of the rigid registration. In addition to displaying the fusion image, the registration reliability of different positions in the registration results is preferably displayed on the basis of the similarity of a plurality of positions in the fusion image. As a display method, when causing the display 22 to display the registration reliability, the display control function 700 presents the level of the reliability in different colors for different positions in the fusion image. By marking, in different colors, positions in the fusion image where deformation is small and the registration reliability is high and positions in the fusion image where deformation is large and the registration reliability is low, the reliability of each position in the fusion image can be intuitively presented to a user. Display examples of the fusion images are described later. The display control function 700 is an example of a “display control unit”.

An example of a registration of an ultrasonic image and an MR image including a prostate is described below.

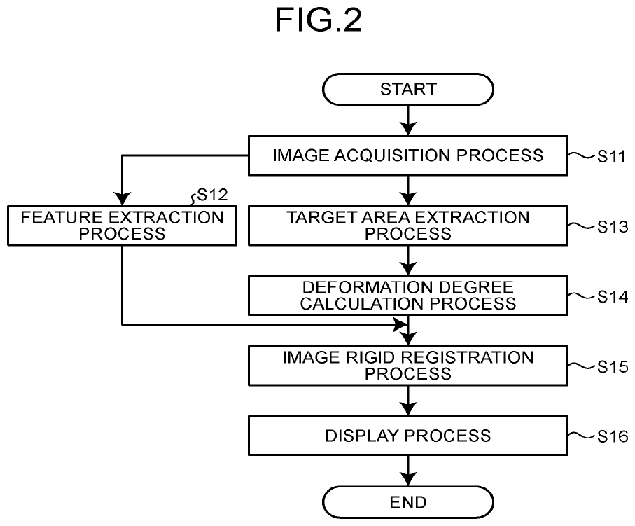

FIG. 2 is a flowchart illustrating a process (medical image processing method) performed by the medical image processing apparatus according to the first embodiment.

An image acquisition process of step S11 in FIG. 2 is a step performed by the processing circuitry 10 calling a computer program corresponding to the image acquisition function 200 from the storage circuitry 100. At step S11, the image acquisition function 200 acquires an ultrasonic image including a prostate and an MR image including the prostate as registration target images. In the present embodiment, the ultrasonic image is an image acquired by an ultrasonic imaging apparatus after the start of surgery, and the MR image is an image acquired in advance by an MR imaging apparatus and stored in the storage circuitry 100.

A feature extraction process at step S12 in FIG. 2 is a step performed by the processing circuitry 10 calling a computer program corresponding to the feature extraction function 300 from the storage circuitry 100. At step S12, the feature extraction function 300 extracts image features from two types of registration target images. The image feature extracted in the present embodiment is a gradient map, and thus a gradient image for each of the two types of registration target images can be obtained.

A target area extraction process of step S13 in FIG. 2 is a step performed by the processing circuitry 10 calling a computer program corresponding to the target area extraction function 400 from the storage circuitry 100. At step S13, the target area extraction function 400 extracts a prostate area corresponding to the prostate (registration target area) from the ultrasonic image by acquiring edges of the prostate, which is a registration target organ, from the ultrasonic image. The extraction method used in the present embodiment is, for example, an edge segmentation algorithm.

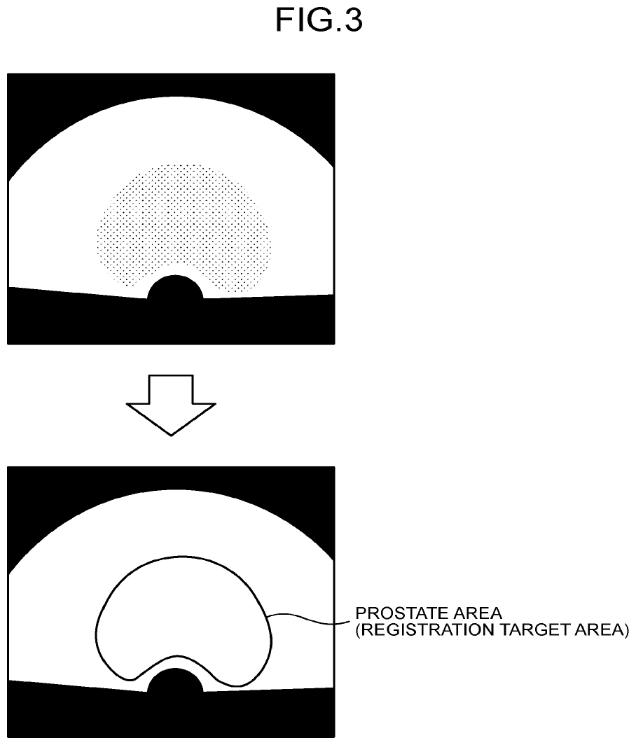

FIG. 3 is a schematic diagram illustrating the extraction of the prostate area (registration target area) from the ultrasonic image. In FIG. 3, an axial view is described as an example, in which an upper part of the drawing is a schematic diagram of the ultrasonic image from which the prostate area is not extracted, and a lower part of the drawing is a schematic diagram of the ultrasonic image from which the prostate area is extracted.

A deformation degree calculation process at step S14 in FIG. 2 is a step performed by the processing circuitry 10 calling a computer program corresponding to the deformation degree calculation function 500 from the storage circuitry 100. At step S14, the deformation degree calculation function 500 calculates the degree of deformation for each position of the prostate area in the ultrasonic image. In the present embodiment, the deformation degree calculation function 500 calculates the degree of deformation for each position by using three types of parameters: the edge unevenness parameter, the edge smoothness parameter, and the probe distance parameter. The deformation degree calculation function 500 also generates an optimal combination of deformation coefficients by adjusting weights of the three types of deformation coefficients according to the state of deformation of the prostate in the ultrasonic image.

The edge unevenness parameter is described below.

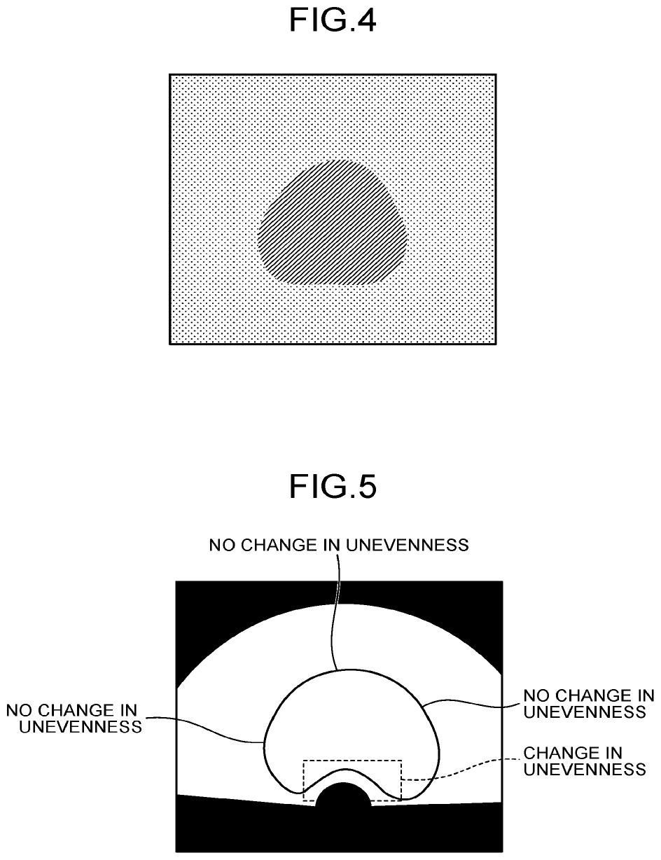

The reason why the degree of deformation is calculated using the unevenness of the edge is because, when the prostate, which is the registration target organ in the present embodiment, is not pressed, the overall contour of the organ in a normal state is a roughly chestnut-shape as illustrated in an axial view of an MR image in FIG. 4, but when the prostate is pressed by an ultrasonic probe and deformed, the unevenness of the edge is changed.

When calculating the edge unevenness coefficient, the positive direction of the organ contour is first specified, and for the prostate in the present embodiment, the positive direction is the direction in which the contour is convex outward. In such a case, an edge unevenness coefficient dᵢ(p) is calculated by Equation 1 below:

$${d}_{1} ( p ) = \{ \begin{matrix}convex ( q ) \cdot {distance ( p , q )}^{- 1} & distance ( p , q ) \leq T \\ C & distance ( p , q ) > T\end{matrix} \qquad ( Equation 1 )$$

where p is any one point in the prostate area and q is an edge point closest to the point p. convex(q) is the unevenness of the point q (1 in the positive direction in which the contour is convex outward and 0 in the negative direction in which the contour is concave inward), and distance(p,q) is the distance between the point p and the point q. T is a preset distance threshold and C is a preset fixed coefficient.

As can be seen from Equation 1 above, the closer the point p is to the edge, the greater the influence received from the unevenness of the edge, but when the distance between the point p and the point q is greater than the distance threshold T, the unevenness of the edge has no influence on the deformation coefficient.

FIG. 5 is a diagram for explaining the edge unevenness of the prostate in the ultrasonic image. In FIG. 5, an area within a dashed frame is a portion that is highly deformed among the edges of the prostate pressed by the ultrasonic probe, and the unevenness of the portion has changed compared to the shape in the normal state. That is, the portion changes from the positive direction in which the contour is convex outward (see FIG. 4) to the negative direction in which the contour is concave inward. On the other hand, portions, other than the dashed frame among the edges of the prostate, are less deformed, and the unevenness of these portions has not changed much compared to the shape in the normal state.

The edge smoothness parameter is described below.

The reason why the degree of deformation is calculated based on the smoothness of the edge is because, when the prostate, which is the registration target organ, is not pressed, the edge of the organ in the normal state is smooth and has a small curvature, but when the organ is deformed by the pressure of the ultrasonic probe, the curvature of the edge increases.

When calculating the edge smoothness coefficient, the edge curvature is added to the deformation coefficient. In such a case, an edge smoothness coefficient d₂(p) is calculated by Equation 2 below:

$${d}_{2} ( p ) = \{ \begin{matrix}curvature ( q ) \cdot {distance ( p , q )}^{- 1} & distance ( p , q ) \leq T \\ C & distance ( p , q ) > T\end{matrix} \qquad ( Equation 2 )$$

where p is any one point in the prostate area and q is an edge point closest to the point p. curvature(q) is the curvature of the point q, and distance(p,q) is the distance between the point p and the point q. T is the preset distance threshold and C is the preset fixed coefficient.

As can be seen from Equation 2 above, the closer the point p is to the edge, the greater the influence received from the edge curvature, but when the distance between the point p and the point q is greater than the distance threshold T, the edge curvature has no influence on the deformation coefficient.

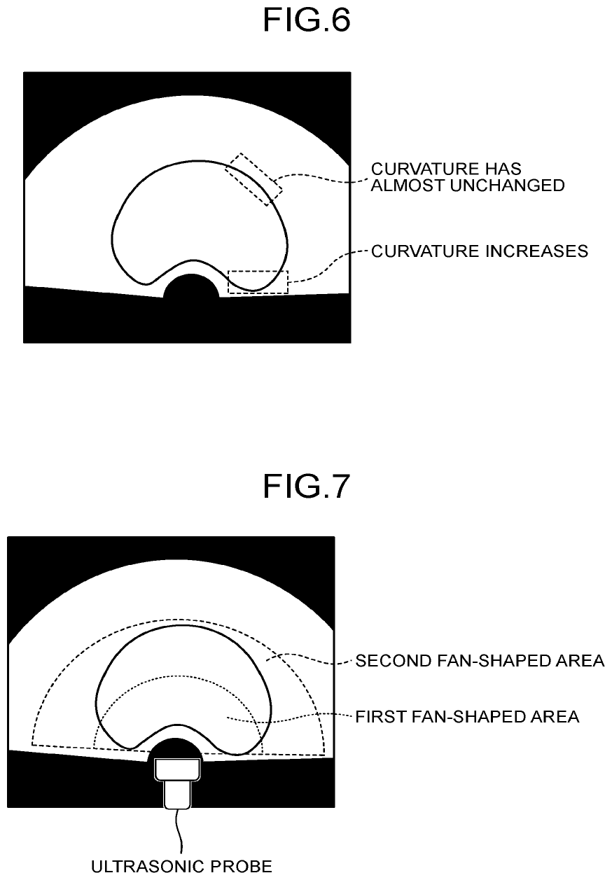

FIG. 6 is a schematic diagram for explaining the relationship between the edge curvature and the deformation of the prostate. An area within a dashed frame near a lower part in FIG. 6 is a portion among the edges of the prostate, which is highly deformed by the pressure of the ultrasonic probe, and the smoothness of the portion has changed compared to the shape in the normal state. That is, the curvature increases. On the other hand, an area within a dashed frame near an upper part in FIG. 6 is a portion that is less deformed among the edges of the prostate pressed by the ultrasonic probe, the smoothness of the portion has almost unchanged and the curvature has almost unchanged compared to the shape in the normal state. FIG. 6 also illustrates that the edge curvature in the dashed frame near the lower part is larger than the edge curvature in the dashed frame near the upper part.

The probe distance parameter is described below.

The reason why the degree of deformation is calculated based on the distance to the probe is because an area closer to the probe is more likely to be pressed and deformed.

When calculating the probe distance coefficient, a large deformation coefficient is given to an area close to the probe and a small deformation coefficient is given to an area far from the probe. In such a case, a probe distance coefficient d₃(p) is calculated by Equation 3 below:

d(p)=decay_function(distance(p,probe)) (Equation 3)

where p is any one point in the prostate area, distance(p,probe) is the distance between the point p and the probe, and decay_function is a decay function. The decay function decay_function may be an exponential decay function e^{k/x} or a power decay function x^{k}, and k is a decay parameter.

As can be seen from Equation 3 above, the closer the point p is to the probe, the greater the influence on the deformation coefficient, but the farther the point p is from the probe, the smaller the influence on the deformation coefficient.

FIG. 7 is a schematic diagram for explaining the relationship between the probe distance and the deformation in the ultrasonic image. In FIG. 7, a first fan-shaped area with a small radius is a portion relatively close to the ultrasonic probe, and the deformation of the portion is large. In FIG. 7, a second fan-shaped area located outside the first fan-shaped area is relatively far from the ultrasonic probe, and the deformation of the portion is small.

In the present embodiment, an optimal combination of deformation coefficients is generated by adjusting the weights of the above three types of deformation coefficients. An optimal combination d(p) of deformation coefficients is calculated by Equation 4 below:

$$d ( p ) = \frac{1}{N} {\sum }_{i ∈ N} {w}_{i} {d}_{i} ( p ) \qquad ( Equation 4 )$$

where dᵢ(p) is an i^{th} kind of deformation coefficient and wᵢ is a weight according to the i^{th} kind of deformation coefficient. The proportion occupied by each deformation coefficient may be set in advance on the basis of empirical values, or may be set on the basis of results of machine learning or the like. For example, weights of the edge unevenness coefficient, the edge smoothness coefficient, and the probe distance coefficient are 0.3, 0.3, and 0.4, respectively.

The description is continued by referring now back to FIG. 2. An image rigid registration process at step S15 in FIG. 2 is a step performed by the processing circuitry 10 calling a computer program corresponding to the image rigid registration function 600 from the storage circuitry 100. At step S15, on the basis of the degree of deformation for each position calculated at the positions and the similarity between the types of medical images at the positions, the image rigid registration function 600 performs rigid registration on the ultrasonic image and the MR image in the present embodiment.

In the registration process, the position of optimal similarity is found by adjusting the amounts of rotation and translation between the images. To reduce the influence of a deformed portion of the registration target organ on the registration, deformation coefficients are added to the similarity calculation, and weighted similarity is obtained by giving a large weight to an area with a small deformation and giving a small weight to an area with a large deformation.

The weighted similarity considering the deformation coefficients is calculated by Equation 5 below:

similarity_w(p,q)=similarity(p,q)·d(p)⁻¹ (Equation 5)

where p is any one point on the ultrasonic image and q is any one point on the MR image. similarity(p,q) is the similarity between the point p and the point q, and d(p) is the deformation coefficient of the point p calculated at the deformation degree calculation step S14.

More specifically, the similarity(p,q) indicates the similarity between any one point p in the feature map of the ultrasonic image and any one point q in the feature map of the MR image. In the present embodiment, since the gradient images of the ultrasonic image and the MR image are obtained at step S12, the similarity(p,q) is, for example, an included angle of a gradient feature vector between the point p in the gradient image of the ultrasonic image and the point q in the gradient image of the MR image.

When the gradient feature vector of the point p is vₚ=(v₁^{p}, v₂^{p}, v₃^{p}) and the gradient feature vector of the point q is v_{q}=(v₁^{q}, v₂^{q}, v₃^{q}), the similarity between the two gradient images is calculated by Equation 6 below.

$$similarity ( p , q ) = \frac{\vert {v}_{1}^{p} {v}_{1}^{q} + {v}_{2}^{p} {v}_{2}^{q} + {v}_{3}^{p} {v}_{3}^{q} \vert}{\vert {v}^{p} \vert \cdot \vert {v}^{q} \vert} \qquad ( Equation 6 )$$

When the feature map is not a gradient image but a gradation map, the similarity(p,q) is, for example, a value of mutual information between the point p in the gradation map of the ultrasonic image and the point q in the gradation map of the MR image.

When the feature map is not a gradient image but a dispersion map, the similarity(p,q) is, for example, a correlation coefficient between the point p in the dispersion map of the ultrasonic image and the point q in the dispersion map of the MR image.

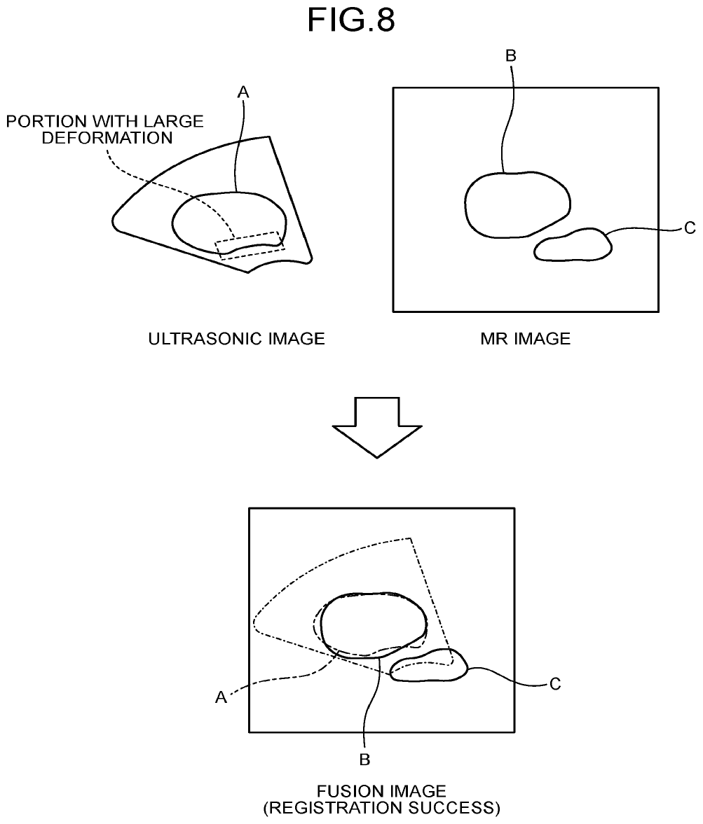

FIG. 8 is a schematic diagram of a successful example of a registration of the ultrasonic image and the MR image according to the first embodiment. In FIG. 8, a sagittal view schematic diagram including the prostate is used as an example.

In FIG. 8, a prostate area A in the ultrasonic image is a prostate area deformed by the pressure of the ultrasonic probe. More specifically, the lower right portion in the prostate area is highly deformed by the pressure of the ultrasonic probe. The highly deformed portion (portion indicated by a dashed frame) changes from an outwardly convex shape before being pressed to an inwardly concave shape.

On the other hand, in FIG. 8, a prostate area B in the MR image is a prostate area where no deformation has occurred. In FIG. 8, the MR image also illustrates a rectal area C.

When the ultrasonic image and the MR image are registered by each step of the present embodiment, the degree of deformation for each position in the ultrasonic image is calculated at step S14, and when the similarity is calculated at step S15, a large weight is given to an area with a small degree of deformation and a small weight is given to an area with a large degree of deformation. Therefore, even though a portion with a large deformation exists in the registration target area, successful registration is achieved and a fusion image in FIG. 8 is obtained as the registration result. In the lower part of FIG. 8, the prostate area A (indicated by the dashed-dotted line) in the ultrasonic image and the prostate area B (indicated by the solid line) in the MR image overlap for the most part, and particularly, a portion with a small deformation (upper half of the prostate area having an approximately oval shape in the fusion image) has high degree of overlap.

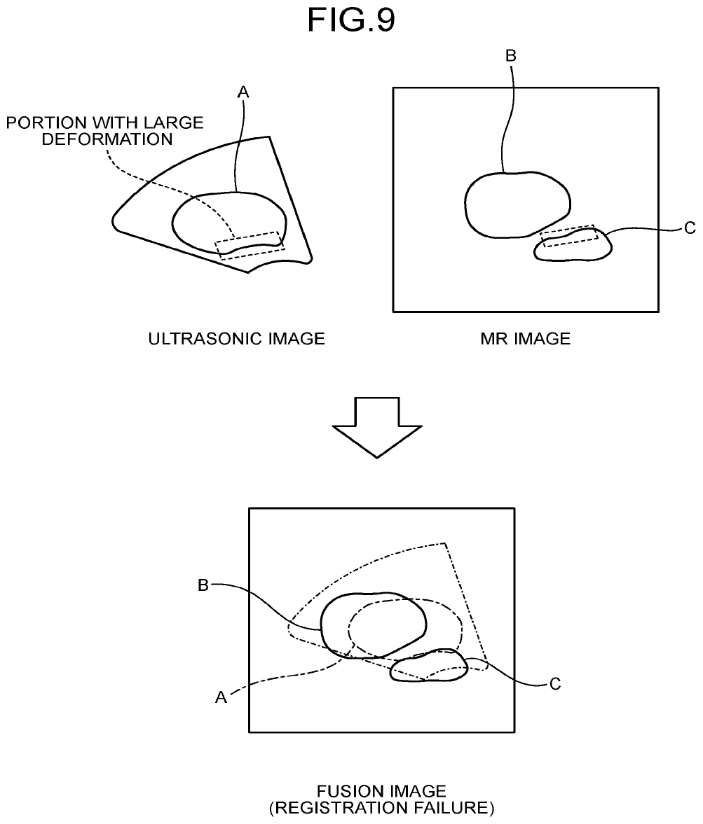

As a comparative example, FIG. 9 illustrates a failure example of a registration of the ultrasonic image and the MR image. In the example illustrated in FIG. 9, a portion with a large deformation in the ultrasonic image is similar to some edges in the rectal area C in the MR image (portion indicated by a dashed frame in the MR image), and in the comparative example, a registration is performed without considering the degree of deformation for each position in the image. This may result in a registration result illustrated in a fusion image in FIG. 9.

In the fusion image in FIG. 9, since edges of the portion with a large deformation in the prostate area A in the ultrasonic image and some edges in the rectal area C (portion indicated by the dashed frame in the MR image) have a high degree of similarity, they may be erroneously registered together. On the other hand, since the prostate area A (indicated by the dashed-dotted line) in the ultrasonic image and the prostate area B (indicated by the solid line) in the MR image do not have nearly overlapping edges and are offset by a certain distance as a whole, the fusion image in FIG. 9 is a fusion image in which registration fails.

The description is continued by referring now back to FIG. 2. A display process of step S16 in FIG. 2 is a step performed by the processing circuitry 10 calling a computer program corresponding to the display control function 700 from the storage circuitry 100. At step S16, the display control function 700 causes the display 22 to display the fusion image representing the registration result that is the result of the rigid registration in FIG. 8.

At step S16, the display control function 700 causes the display 22 to display the fusion image, and also preferably causes the display 22 to display the registration reliability of different positions in the registration result on the basis of the similarity of a plurality of positions in the fusion image. For example, when displaying the registration reliability, the display control function 700 presents the level of the reliability in different colors for different positions in the fusion image.

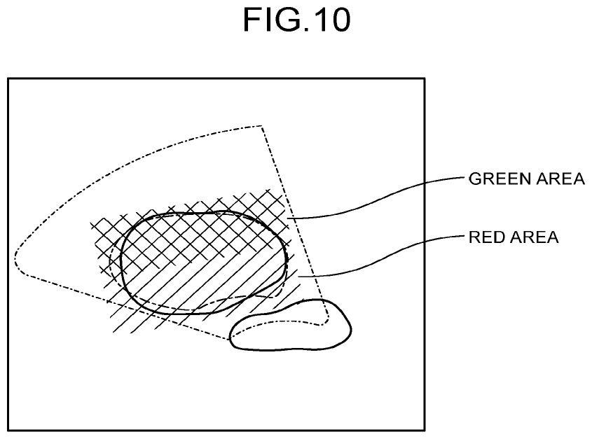

FIG. 10 is a diagram illustrating a display example of the registration reliability. In the fusion image illustrated in FIG. 10, areas with small deformation and high registration reliability are marked, for example, in green (indicated by diamond patterns in the drawing), and areas with large deformation and low registration reliability are marked, for example, in red (indicated by diagonal lines in the drawing). This allows the display control function 700 to intuitively present a user with reliability corresponding to different areas in the fusion image by causing the display 22 to display the fusion image illustrated in FIG. 10. In addition to the color, the areas may be distinguished by different figures or patterns.

Effects of the medical image processing apparatus 1 according to the first embodiment are described below.

First, the medical image processing apparatus 1 according to the first embodiment calculates the degree of deformation of a plurality of positions in a registration target area in an ultrasonic image, and performs a rigid registration on a plurality of types of medical images on the basis of the degree of deformation for each position calculated at the positions and the similarity between the types of medical images at the positions. Thus, in the present embodiment, even though the deformation of a registration target organ included in the ultrasonic image is large, interference of a portion with a large deformation with the rigid registration can be suppressed, and accuracy of the rigid registration of the ultrasonic image and an MR image can be improved.

In the present embodiment, the rigid registration is performed by calculating weighted similarity so that a large weight is given to an area with a small deformation and a small weight is given to an area with a large deformation. Thus, in the present embodiment, adverse effects on a registration result of an area with a large degree of deformation can be automatically reduced, contribution to a registration result of an area with no deformation or a small degree of deformation can be reinforced, and interference of the portion with a large deformation in the ultrasonic image with the rigid registration is more reliably suppressed.

Furthermore, in the present embodiment, by selecting at least one of the edge unevenness parameter, the edge smoothness parameter, and the probe distance parameter, the accuracy of the calculated degree of deformation can be improved by using deformation parameters according to the features of the registration target organ. Moreover, in the present embodiment, an optimum combination of deformation coefficients is generated by adjusting the weights of two or more types of coefficients according to the state of deformation of the registration target organ. Thus, in the present embodiment, deformation parameters more adapted to the features of the registration target organ can be obtained.

### Second Embodiment

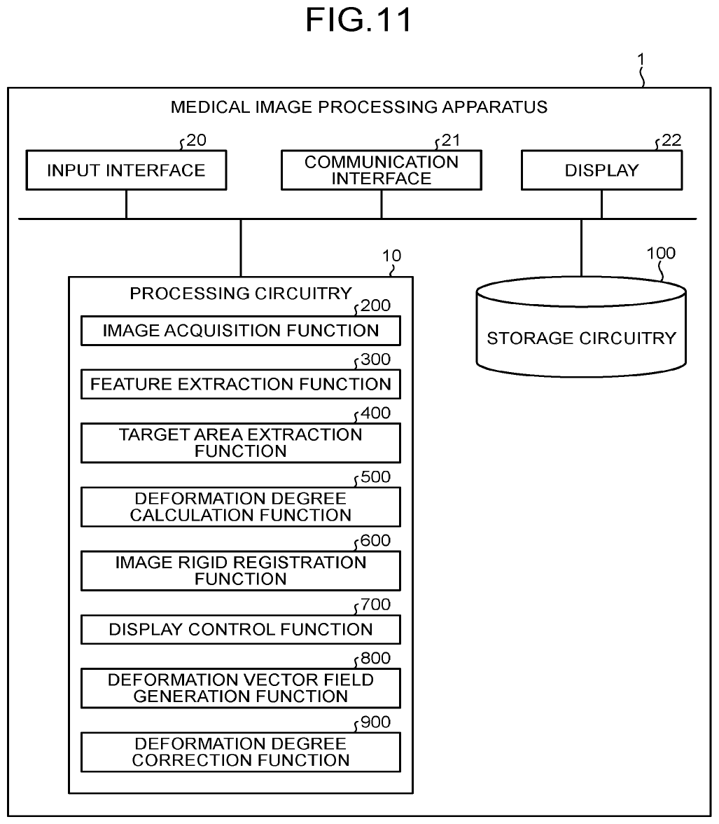

A medical image processing apparatus 1 according to a second embodiment is described below with reference to FIGS. 11 to 13.

FIG. 11 is a block diagram illustrating an example of the configuration of the medical image processing apparatus 1 according to the second embodiment. As illustrated in FIG. 11, the medical image processing apparatus 1 includes a deformation vector field generation function 800 and a deformation degree correction function 900 in addition to the functions illustrated in FIG. 1. The same descriptions as those of the first embodiment are omitted, and only different points are described below.

The deformation vector field generation function 800 performs a non-rigid registration on a plurality of types of medical images (two types of medical images in the present embodiment) at a plurality of positions to generate deformation vector fields. A deformation vector field between two types of medical images can be obtained using a non-rigid registration algorithm (for example, an ICP algorithm or the like) in the related art. The deformation vector field generation function 800 is an example of a “deformation vector field generation unit”.

The deformation degree correction function 900 corrects the degree of deformation at the positions, which is calculated by the deformation degree calculation function 500, on the basis of the magnitudes of vectors in the deformation vector fields corresponding to the two types of medical images, so that as the magnitude of the vector increases, the degree of deformation increases. The deformation degree correction function 900 is an example of a “deformation degree correction unit”.

The image rigid registration function 600 in the second embodiment performs the rigid registration again on the types of medical images on the basis of the degree of deformation for each position corrected at the positions and the similarity between the two types of medical images at the positions.

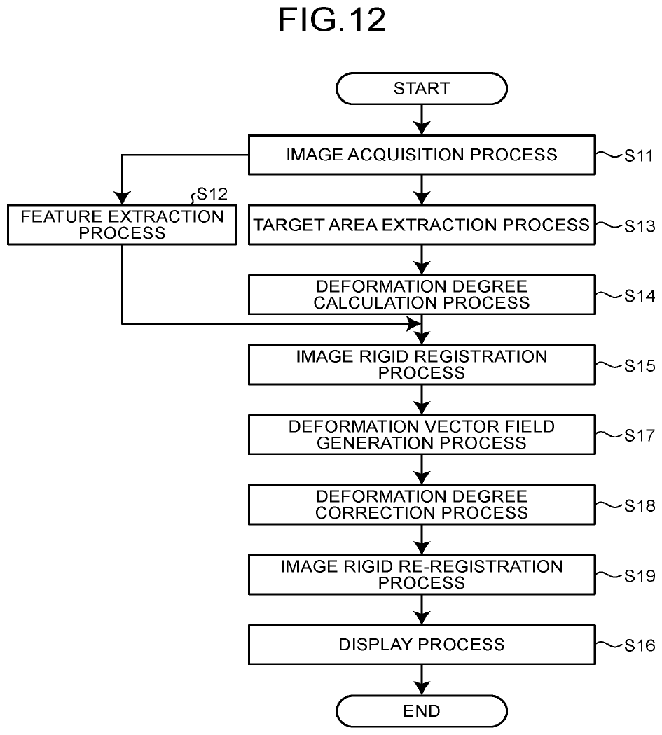

FIG. 12 is a flowchart illustrating a process (medical image processing method) performed by the medical image processing apparatus according to the second embodiment.

In FIG. 12, steps S17 to S19 are added after step S15. A deformation vector field generation process at step S17 in FIG. 12 is a step performed by the processing circuitry 10 calling a computer program corresponding to the deformation vector field generation function 800 from the storage circuitry 100. At step S17, the deformation vector field generation function 800 performs the non-rigid registration on the ultrasonic image and the MR image at the positions to generate the deformation vector fields. The deformation vector fields from the ultrasonic image to the MR image can be obtained using the non-rigid registration algorithm (for example, an ICP algorithm or the like) in the related art.

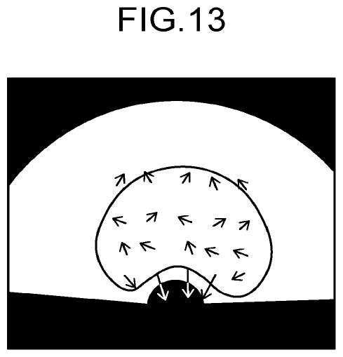

FIG. 13 is a schematic diagram illustrating the relationship between vectors in the deformation vector field and the degree of deformation in the second embodiment. As illustrated in FIG. 13, in an area with a small deformation (that is, area near an upper part in a prostate area in the drawing), the magnitude of each vector is small, and in an area with a large deformation (that is, area near a lower part in the prostate area in the drawing), the magnitude of each vector (particularly, some vectors at the lower part) is large.

A deformation degree correction process at step S18 in FIG. 12 is a step performed by the processing circuitry 10 calling a computer program corresponding to the deformation degree correction function 900 from the storage circuitry 100. At step S18, the deformation degree correction function 900 corrects the degree of deformation previously calculated at the positions, on the basis of the magnitudes of the vectors in the deformation vector fields corresponding to the ultrasonic image and the MR image, so that as the magnitude of the vector increases, the degree of deformation increases.

As an example, the deformation degree correction function 900 corrects the optimal combination d(p) of deformation coefficients calculated at the deformation degree calculation step S14, according to Equation 7 below:

d(p)=d(p)+λ·|transform_field(p)| (Equation 7)

where p is any one point in the ultrasonic image, |transform field(P)| is the magnitude of the deformation vector field at the point p, and λ is a weight coefficient. The weight coefficient λ may be set in advance on the basis of empirical values or may be set on the basis of results of machine learning or the like. For example, the weight coefficient λ can be selected in the range of [0.01 to 0.2].

An image rigid re-registration process at step S19 in FIG. 12 is a step performed by the processing circuitry 10 calling a computer program corresponding to the image rigid registration function 600 from the storage circuitry 100. At step S19, the image rigid registration function 600 performs the rigid registration again on the ultrasonic image and the MR image on the basis of the degree of deformation for each position corrected at the positions and the similarity between the two types of medical images at the positions. At step S19, the weighted similarity is calculated again on the basis of the degree of deformation corrected based on the first rigid registration, and the position of the optimal similarity is further found by adjusting the amounts of rotation and translation between the two medical images in a small range.

In this way, the medical image processing apparatus 1 according to the second embodiment generates the deformation vector field by the non-rigid registration and corrects the optimal combination d(p) of deformation coefficients by using the magnitude of a deformation amount represented by the magnitude of each vector. Thus, in the present embodiment, a more accurate optimal combination d(p) of deformation coefficients can be obtained, and the accuracy of image registration can be further improved.

### Other Variations

Although some embodiments have been described, the above-described embodiments are presented as examples and are not intended to limit the scope of the invention. These embodiments can be implemented in various other forms, and various omissions, substitutions, and changes can be made without departing from the gist of the invention. These embodiments and variations thereof fall within the scope and gist of the invention and within the scope of the invention defined in the technical proposal and equivalents thereof.

For example, each of the above-described embodiments is described using, as an example, a registration of an ultrasonic image and an MR image including a prostate; however, it is of course that the embodiments can be applied to a case of a registration of an ultrasonic image and a CT image.

In the above-described first and second embodiments, the fusion image is displayed at step S16 as a result of the registration at step S15 or step S19; however, the embodiments are not limited thereto. For example, in the process at step S16, not only the fusion image but also an image obtained by further fusing the fusion image with another medical image may be displayed. Instead of displaying the fusion image, the fusion image may be further analyzed and the result of the analysis process may be displayed.

Step S16 may not be performed, and as a result of the registration at step S15 or step S19, a correspondence relation between an ultrasonic image coordinate system and an MR image coordinate system is obtained. This correspondence relation is stored and used, for example, in the form of a transformation matrix.

According to at least one of the above-described embodiments, even though a registration target organ included in an ultrasonic image is highly deformed, a registration of the ultrasonic image and other types of medical images can be achieved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising:
processing circuitry configured to
extract a registration target area corresponding to a registration target organ from an ultrasonic image by acquiring edges of the registration target organ from the ultrasonic image among a plurality of types of medical images including the registration target organ,
calculate a degree of deformation of a plurality of positions in the registration target area, and
perform a rigid registration on the types of medical images on the basis of the degree of deformation for each position calculated at the positions and similarity between the types of medical images at the positions.

2. The medical image processing apparatus according to claim 1, wherein the processing circuitry causes a display to
display a fusion image representing a registration result that is a result of the rigid registration, and
display registration reliability of different positions in the registration result on the basis of the similarity of the positions in the fusion image.

3. The medical image processing apparatus according to claim 1, wherein each position in the positions is a sub-area including one pixel point or a plurality of pixel points in the registration target area.

4. The medical image processing apparatus according to claim 1, wherein
the degree of deformation includes at least one of a parameter indicating unevenness of an edge, a parameter indicating smoothness of the edge, and a parameter indicating a distance from an organ to a pressing object, and
when the degree of deformation includes two or more types of parameters, the processing circuitry generates an optimal combination of deformation coefficients by adjusting weights of the two or more types of coefficients according to a state of deformation of the registration target organ.

5. The medical image processing apparatus according to claim 1, wherein the processing circuitry calculates weighted similarity so that a large weight is given to an area with a small deformation and a small weight is given to an area with a large deformation, on the basis of the degree of deformation for each position calculated at the positions and the similarity between the types of medical images at the positions, and performs the rigid registration on the types of medical images on the basis of the weighted similarity.

6. The medical image processing apparatus according to claim 2, wherein, when causing the display to display the registration reliability, the processing circuitry presents a level of the reliability in different colors for different positions in the fusion image.

7. The medical image processing apparatus according to claim 1, wherein the processing circuitry
performs, after the rigid registration is performed, a non-rigid registration on the types of medical images at the positions to generate deformation vector fields,
corrects the degree of deformation of the positions on the basis of magnitudes of vectors in the deformation vector fields corresponding to the types of medical images so that as a magnitude of the vector increases, the degree of deformation increases, and
performs the rigid registration again on the types of medical images on the basis of the degree of deformation for each position corrected at the positions and the similarity between the types of medical images at the positions.

8. A medical image processing method comprising:
extracting a registration target area corresponding to a registration target organ from an ultrasonic image by acquiring edges of the registration target organ from the ultrasonic image among a plurality of types of medical images including the registration target organ,
calculating a degree of deformation of a plurality of positions in the registration target area; and
performing a rigid registration on the types of medical images on the basis of the degree of deformation for each position calculated at the positions and similarity between the types of medical images at the positions.

9. A non-transitory computer readable medium comprising instructions that cause a computer to execute:
extracting a registration target area corresponding to a registration target organ from an ultrasonic image by acquiring edges of the registration target organ from the ultrasonic image among a plurality of types of medical images including the registration target organ,
calculating a degree of deformation of a plurality of positions in the registration target area; and
performing a rigid registration on the types of medical images on the basis of the degree of deformation for each position calculated at the positions and similarity between the types of medical images at the positions.
